(19) United States
(12) Patent Application Publication
(10) Pub. No.: US 2020/0054304 A1
(43) Pub. Date: Feb. 20, 2020

(54) MOLDED TIP WITH EXTENDED GUIDEWIRE LUMEN AND ASSOCIATED DEVICES, SYSTEMS, AND METHODS

(71) Applicant: KONINKLIJKE PHILIPS N.V., Eindhoven (NL)

(72) Inventors: Jeremy STIGALL, Carlsbad, CA (US); Maritess MINAS, San Diego, CA (US)

(21) Appl. No.: 16/531,687

(22) Filed: Aug. 5, 2019

Related U.S. Application Data
(60) Provisional application No. 62/718,713, filed on Aug. 14, 2018.

(51) Int. Cl. A61B 8/12; A61B 8/00; A61M 25/00; A61M 25/09

(52) U.S. Cl. CPC A61B 8/12; A61B 8/445; A61B 8/462; A61M 2025/0183; A61M 25/09; A61M 25/001; A61B 2562/12; A61M 25/0068

(57) ABSTRACT

Improved intraluminal imaging devices and methods of manufacturing the devices are provided. In one embodiment, an intraluminal imaging device includes a flexible elongate member configured to be positioned within a body lumen of a patient, the flexible elongate member comprising a proximal portion and a distal portion, an imaging assembly coupled to the distal portion of the flexible elongate member, the imaging assembly surrounding a lumen, and a tip member coupled to the imaging assembly, the tip member comprising a molded body including a guiding portion and an extended portion. The guiding portion extends distally of the imaging assembly, and the extended portion extends proximally of the guiding portion through the lumen within the imaging assembly. The tip member comprises a guidewire lumen extending through the guiding portion and the extended portion.

### CROSS-REFERENCE TO PRIOR APPLICATIONS

This application claims the benefit of U.S. Provisional Application No. 62/718,713, filed on 14 Aug. 2018. This application is hereby incorporated by reference herein.

### TECHNICAL FIELD

The present disclosure relates generally to intraluminal medical imaging and, in particular, to the distal structure of an intraluminal imaging device. For example, the distal structure can include a flexible substrate that is rolled onto a support structure, joined to a flexible elongate member, and coated with a film to facilitate efficient assembly and operation of the intravascular imaging device.

### BACKGROUND

Intravascular ultrasound (IVUS) imaging is widely used in interventional cardiology as a diagnostic tool for assessing a diseased vessel, such as an artery, within the human body to determine the need for treatment, to guide the intervention, and/or to assess its effectiveness. An IVUS device including one or more ultrasound transducers is passed into the vessel and guided to the area to be imaged. The transducers emit ultrasonic energy in order to create an image of the vessel of interest. Ultrasonic waves are partially reflected by discontinuities arising from tissue structures (such as the various layers of the vessel wall), red blood cells, and other features of interest. Echoes from the reflected waves are received by the transducer and passed along to an IVUS imaging system. The imaging system processes the received ultrasound echoes to produce a cross-sectional image of the vessel where the device is placed.

Solid-state (also known as synthetic-aperture) IVUS catheters are one of the two types of IVUS devices commonly used today, the other type being the rotational IVUS catheter. Solid-state IVUS catheters carry a scanner assembly that includes an array of ultrasound transducers distributed around its circumference along with one or more integrated circuit controller chips mounted adjacent to the transducer array. The controllers select individual acoustic elements (or groups of elements) for transmitting an ultrasound pulse and for receiving the ultrasound echo signal. By stepping through a sequence of transmit-receive pairs, the solid-state IVUS system can synthesize the effect of a mechanically scanned ultrasound transducer but without moving parts (hence the solid-state designation). Since there is no rotating mechanical element, the transducer array can be placed in direct contact with the blood and vessel tissue with minimal risk of vessel trauma. Furthermore, because there is no rotating element, the electrical interface is simplified. The solid-state scanner can be wired directly to the imaging system with a simple electrical cable and a standard detachable electrical connector, rather than the complex rotating electrical interface required for a rotational IVUS device.

Manufacturing IVUS devices that can efficiently traverse anatomic structures within the human body is challenging. Methods for coupling the various components of the IVUS devices to one another, including adhesives and thermal bonding, can undesirably lead to an increased outer profile of the device and damage to sensitive electronic components of the imaging assembly.

### SUMMARY

Embodiments of the present disclosure provide improved intraluminal imaging devices and methods of manufacturing the devices that overcome the limitations described above. For example, an intraluminal imaging device can include a tip member with an extended guidewire lumen coupled to a distal portion of a flexible elongate member.

In one embodiment, an intraluminal imaging device includes a flexible elongate member configured to be positioned within a body lumen of a patient, the flexible elongate member comprising a proximal portion and a distal portion, an imaging assembly coupled to the distal portion of the flexible elongate member, the imaging assembly surrounding a lumen, and a tip member coupled to the imaging assembly, the tip member comprising a molded body including a guiding portion and an extended portion. The guiding portion extends distally of the imaging assembly, and the extended portion extends proximally of the guiding portion through the lumen within the imaging assembly. The tip member comprises a guidewire lumen extending through the guiding portion and the extended portion.

In some embodiments, the imaging device further comprises an adhesive fillet positioned around an external surface of a proximal portion of the guiding portion, the adhesive fillet contacting a distal end of the imaging assembly such that the fillet seals a junction between the guiding portion of the tip member and the distal end of the imaging assembly. In some embodiments, the imaging assembly comprises an intravascular ultrasound (IVUS) imaging assembly, and the IVUS imaging assembly comprises a flexible substrate positioned around a support member. The imaging assembly can further include an extension tube attached to a proximal flange of the support member, and wherein a proximal end of the extended portion of the tip member is attached to the extension tube. In some embodiments, the flexible substrate comprises an electrical interface disposed at a proximal end of the flexible substrate, and the electrical interface is secured to an outer surface of the extension tube.

In some aspects, the tip member comprises an intermediate connection portion between the guiding portion and the extended portion, the intermediate connection portion comprising a recess extending distally into the guiding portion, and a distal flange of the support member is received within the recess. According to some embodiments, the flexible elongate member comprises a guidewire exit port, and the extended portion extends proximally within the flexible elongate member to the guidewire exit port, such that the guidewire lumen extends from the guidewire exit port to a distal end of the tip member. In some embodiments, the flexible elongate member comprises a proximal inner member and a proximal outer member, and the proximal end of the extended portion of the tip member is coupled to a distal end of the proximal inner member. In some embodiments, the extended portion comprises a radial projection at a proximal end of the extended portion of the tip member, the radial projection configured to engage a proximal surface of the imaging assembly to mechanically secure the tip member to the imaging assembly. In some embodiments, the guiding portion of the tip member comprises a tapered tubular shape comprising a first outer diameter at a proximal end of the guiding portion and a second outer diameter at a distal end of the guiding portion, and wherein the extended portion comprises a non-tapered shape comprising a third outer diameter, wherein the first outer diameter is larger than the second outer diameter and the third outer diameter.

According to some aspects of the present disclosure, a method for manufacturing an intraluminal imaging device includes providing a tip member comprising a molded body including a guiding portion, an extended portion extending proximally of the guiding portion, and an intermediate connection portion disposed at a junction of the guiding portion and the extended portion, positioning the extended portion within a lumen of an imaging assembly, applying adhesive on or near the intermediate connection portion, and moving the tip member proximally such that the intermediate connection portion abuts a distal end of the imaging assembly, and such that a proximal end of the extended portion extends to a proximal portion of the imaging assembly.

In some aspects, positioning the adhesive comprises forming an adhesive fillet on an exterior surface of the intermediate connection portion such that the fillet provides a seal between the guiding portion of the tip member and the imaging assembly. In some embodiments, the intermediate connection portion of the tip member comprises a recess extending distally into the guiding portion, and moving the tip member proximally comprises inserting a distal flange of the imaging assembly into the recess. In some embodiments, moving the tip member proximally comprises positioning a proximal end of the extended portion adjacent a proximal flange of the imaging assembly. In some embodiments, the method further includes at least one of thermally or adhesively bonding an extension around the proximal flange of the imaging assembly and the proximal end of the extended portion. In some embodiments, the method further comprises coupling a distal end of a flexible elongate member to the imaging assembly, and moving the tip member proximally comprises positioning a proximal end of the extended portion at a guidewire exit port of the flexible elongate member.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic schematic view of an intraluminal imaging system, according to aspects of the present disclosure.

FIG. 2 is a diagrammatic perspective view of the top of a scanner assembly in a flat configuration, according to aspects of the present disclosure.

FIG. 3 is a diagrammatic perspective view of the scanner assembly shown in FIG. 2 in a rolled configuration around a support member, according to aspects of the present disclosure.

FIG. 4 is a diagrammatic cross-sectional side view of a conventional scanner assembly with a flexible tip member disposed at a distal end.

FIG. 5 is a cross-sectional side view of a flexible tip member including an extended guidewire lumen, according to aspects of the present disclosure.

FIG. 6 is a diagrammatic cross-sectional side view of the scanner assembly including the flexible tip member shown in FIG. 5, according to aspects of the present disclosure.

FIG. 7 is a diagrammatic cross-sectional side view of a distal portion of an intraluminal imaging device, according to aspects of the present disclosure.

FIG. 8 is a diagrammatic cross-sectional side view of a distal portion of an intraluminal imaging device, according to aspects of the present disclosure.

FIG. 9 is a diagrammatic cross-sectional side view of a distal portion of an intraluminal imaging device, according to aspects of the present disclosure.

FIG. 10 is a flow diagram of a method of manufacturing an intraluminal imaging device, according to aspects of the present disclosure.

FIGS. 11A, 11B, 11C and 11D are perspective views of a scanner assembly and a flexible tip member at various stages of an assembly process, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. For example, while the focusing system is described in terms of cardiovascular imaging, it is understood that it is not intended to be limited to this application. The system is equally well suited to any application requiring imaging within a confined cavity. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

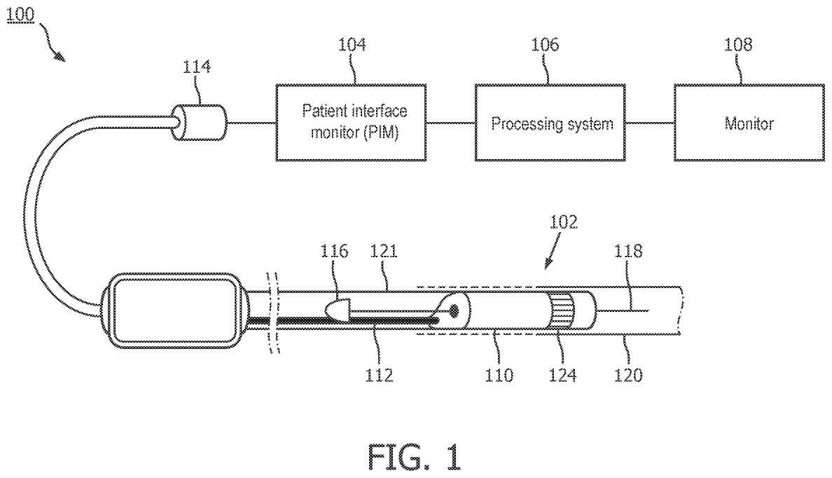

FIG. 1 is a diagrammatic schematic view of an intraluminal imaging system 100, according to aspects of the present disclosure. The intraluminal imaging system 100 can be an ultrasound imaging system. In some instances, the system 100 can be an intravascular ultrasound (IVUS) imaging system. The system 100 may include an intraluminal imaging device 102 such as a catheter, guide wire, or guide catheter, a patient interface module (PIM) 104, an processing system or console 106, and a monitor 108. The intraluminal imaging device 102 can be an ultrasound imaging device. In some instances, the device 102 can be IVUS imaging device, such as a solid-state IVUS device.

At a high level, the IVUS device 102 emits ultrasonic energy from a transducer array 124 included in scanner assembly 110 mounted near a distal end of the catheter device. The ultrasonic energy is reflected by tissue structures in the medium, such as a vessel 120, or another body lumen surrounding the scanner assembly 110, and the ultrasound echo signals are received by the transducer array 124. In that regard, the device 102 can be sized, shaped, or otherwise configured to be positioned within the body lumen of a patient. The PIM 104 transfers the received echo signals to the console or computer 106 where the ultrasound image (including the flow information) is reconstructed and displayed on the monitor 108. The console or computer 106 can include a processor and a memory. The computer or computing device 106 can be operable to facilitate the features of the IVUS imaging system 100 described herein. For example, the processor can execute computer readable instructions stored on the non-transitory tangible computer readable medium.

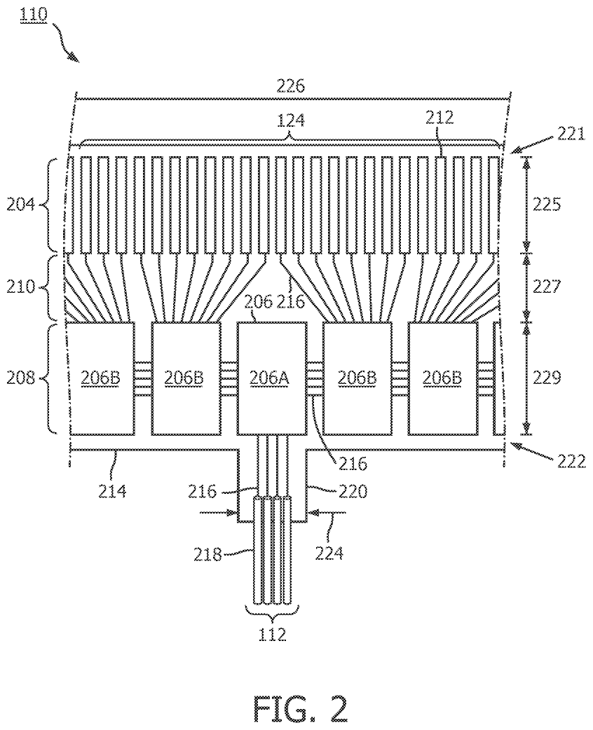

The PIM 104 facilitates communication of signals between the IVUS console 106 and the scanner assembly 110 included in the IVUS device 102. This communication includes the steps of: (1) providing commands to integrated circuit controller chip(s) 206A, 206B, illustrated in FIG. 2, included in the scanner assembly 110 to select the particular transducer array element(s), or acoustic element(s), to be used for transmit and receive, (2) providing the transmit trigger signals to the integrated circuit controller chip(s) 206A, 206B included in the scanner assembly 110 to activate the transmitter circuitry to generate an electrical pulse to excite the selected transducer array element(s), and/or (3) accepting amplified echo signals received from the selected transducer array element(s) via amplifiers included on the integrated circuit controller chip(s)126 of the scanner assembly 110. In some embodiments, the PIM 104 performs preliminary processing of the echo data prior to relaying the data to the console 106. In examples of such embodiments, the PIM 104 performs amplification, filtering, and/or aggregating of the data. In an embodiment, the PIM 104 also supplies high- and low-voltage DC power to support operation of the device 102 including circuitry within the scanner assembly 110.

The IVUS console 106 receives the echo data from the scanner assembly 110 by way of the PIM 104 and processes the data to reconstruct an image of the tissue structures in the medium surrounding the scanner assembly 110. The console 106 outputs image data such that an image of the vessel 120, such as a cross-sectional image of the vessel 120, is displayed on the monitor 108. Vessel or lumen 120 may represent fluid filled or surrounded structures, both natural and man-made. The lumen 120 may be within a body of a patient. The lumen 120 may be a blood vessel, as an artery or a vein of a patient's vascular system, including cardiac vasculature, peripheral vasculature, neural vasculature, renal vasculature, and/or or any other suitable lumen inside the body. For example, the device 102 may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. In addition to natural structures, the device 102 may be may be used to examine man-made structures such as, but without limitation, heart valves, stents, shunts, filters and other devices.

In some embodiments, the IVUS device includes some features similar to traditional solid-state IVUS catheters, such as the EagleEye® catheter available from Volcano Corporation and those disclosed in U.S. Pat. No. 7,846,101 hereby incorporated by reference in its entirety. For example, the IVUS device 102 includes the scanner assembly 110 near a distal end of the device 102 and a transmission line bundle 112 extending along the longitudinal body of the device 102. The transmission line bundle or cable 112 can include a plurality of conductors, including one, two, three, four, five, six, seven, or more conductors 218 (FIG. 2). It is understood that any suitable gauge wire can be used for the conductors 218. In an embodiment, the cable 112 can include a four-conductor transmission line arrangement with, e.g., 41 AWG gauge wires. In an embodiment, the cable 112 can include a seven-conductor transmission line arrangement utilizing, e.g., 44 AWG gauge wires. In some embodiments, 43 AWG gauge wires can be used.

The transmission line bundle 112 terminates in a PIM connector 114 at a proximal end of the device 102. The PIM connector 114 electrically couples the transmission line bundle 112 to the PIM 104 and physically couples the IVUS device 102 to the PIM 104. In an embodiment, the IVUS device 102 further includes a guide wire exit port 116. Accordingly, in some instances the IVUS device is a rapid-exchange catheter. The guide wire exit port 116 allows a guide wire 118 to be inserted towards the distal end in order to direct the device 102 through the vessel 120.

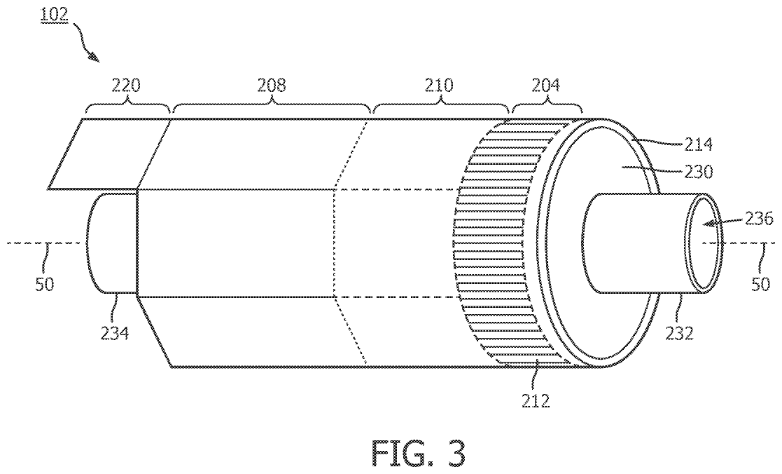

FIG. 3 is a diagrammatic top view of a portion of a flexible assembly 200, according to aspects of the present disclosure. The flexible assembly 200 includes a transducer array 124 formed in a transducer region 204 and transducer control logic dies 206 (including dies 206A and 206B) formed in a control region 208, with a transition region 210 disposed therebetween. The transducer array 124 includes an array of ultrasound transducers 212. The transducer control logic dies 206 are mounted on a flexible substrate 214 into which the transducers 212 have been previously integrated. The flexible substrate 214 is shown in a flat configuration in FIG. 2. Though six control logic dies 206 are shown in FIG. 2, any number of control logic dies 206 may be used. For example, one, two, three, four, five, six, seven, eight, nine, ten, or more control logic dies 206 may be used.

The flexible substrate 214, on which the transducer control logic dies 206 and the transducers 212 are mounted, provides structural support and interconnects for electrical coupling. The flexible substrate 214 may be constructed to include a film layer of a flexible polyimide material such as KAPTON™ (trademark of DuPont). Other suitable materials include polyester films, polyimide films, polyethylene napthalate films, or polyetherimide films, liquid crystal polymer, other flexible printed semiconductor substrates as well as products such as Upilex® (registered trademark of Ube Industries) and TEFLON® (registered trademark of E.I. du Pont). In the flat configuration illustrated in FIG. 2, the flexible substrate 214 has a generally rectangular shape. As shown and described herein, the flexible substrate 214 is configured to be wrapped around a support member 230 (FIG. 3) in some instances. Therefore, the thickness of the film layer of the flexible substrate 214 is generally related to the degree of curvature in the final assembled flexible assembly 110. In some embodiments, the film layer is between 5 μm and 100 μm, with some particular embodiments being between 5 μm and 25.1 m, e.g., 6 μm.

The transducer control logic dies 206 is a non-limiting example of a control circuit. The transducer region 204 is disposed at a proximal portion 221 of the flexible substrate 214. The control region 208 is disposed at a proximal portion 222 of the flexible substrate 214. The transition region 210 is disposed between the control region 208 and the transducer region 204. Dimensions of the transducer region 204, the control region 208, and the transition region 210 (e.g., lengths 225, 227, 229) can vary in different embodiments. In some embodiments, the lengths 225, 227, 229 can be substantially similar or, the length 227 of the transition region 210 may be less than lengths 225 and 229, the length 227 of the transition region 210 can be greater than lengths 225, 229 of the transducer region and controller region, respectively.

The control logic dies 206 are not necessarily homogenous. In some embodiments, a single controller is designated a master control logic die 206A and contains the communication interface for cable 142 which may serve as an electrical conductor, e.g., electrical conductor 112, between a processing system, e.g., processing system 106, and the flexible assembly 200. Accordingly, the master control circuit may include control logic that decodes control signals received over the cable 142, transmits control responses over the cable 142, amplifies echo signals, and/or transmits the echo signals over the cable 142. The remaining controllers are slave controllers 206B. The slave controllers 206B may include control logic that drives a transducer 212 to emit an ultrasonic signal and selects a transducer 212 to receive an echo. In the depicted embodiment, the master controller 206A does not directly control any transducers 212. In other embodiments, the master controller 206A drives the same number of transducers 212 as the slave controllers 206B or drives a reduced set of transducers 212 as compared to the slave controllers 206B. In an exemplary embodiment, a single master controller 206A and eight slave controllers 206B are provided with eight transducers assigned to each slave controller 206B.

To electrically interconnect the control logic dies 206 and the transducers 212, in an embodiment, the flexible substrate 214 includes conductive traces 216 formed in the film layer that carry signals between the control logic dies 206 and the transducers 212. In particular, the conductive traces 216 providing communication between the control logic dies 206 and the transducers 212 extend along the flexible substrate 214 within the transition region 210. In some instances, the conductive traces 216 can also facilitate electrical communication between the master controller 206A and the slave controllers 206B. The conductive traces 216 can also provide a set of conductive pads that contact the conductors 218 of cable 142 when the conductors 218 of the cable 142 are mechanically and electrically coupled to the flexible substrate 214. Suitable materials for the conductive traces 216 include copper, gold, aluminum, silver, tantalum, nickel, and tin, and may be deposited on the flexible substrate 214 by processes such as sputtering, plating, and etching. In an embodiment, the flexible substrate 214 includes a chromium adhesion layer. The width and thickness of the conductive traces 216 are selected to provide proper conductivity and resilience when the flexible substrate 214 is rolled. In that regard, an exemplary range for the thickness of a conductive trace 216 and/or conductive pad is between 1-5 m. For example, in an embodiment, 5 m conductive traces 216 are separated by m of space. The width of a conductive trace 216 on the flexible substrate may be further determined by the width of the conductor 218 to be coupled to the trace/pad.

The flexible substrate 214 can include a conductor interface 220 in some embodiments. The conductor interface 220 can be a location of the flexible substrate 214 where the conductors 218 of the cable 142 are coupled to the flexible substrate 214. For example, the bare conductors of the cable 142 are electrically coupled to the flexible substrate 214 at the conductor interface 220. The conductor interface 220 can be tab extending from the main body of flexible substrate 214. In that regard, the main body of the flexible substrate 214 can refer collectively to the transducer region 204, controller region 208, and the transition region 210. In the illustrated embodiment, the conductor interface 220 extends from the proximal portion 222 of the flexible substrate 214. In other embodiments, the conductor interface 220 is positioned at other parts of the flexible substrate 214, such as the proximal portion 221, or the flexible substrate 214 may lack the conductor interface 220. A value of a dimension of the tab or conductor interface 220, such as a width 224, can be less than the value of a dimension of the main body of the flexible substrate 214, such as a width 226. In some embodiments, the substrate forming the conductor interface 220 is made of the same material(s) and/or is similarly flexible as the flexible substrate 214. In other embodiments, the conductor interface 220 is made of different materials and/or is comparatively more rigid than the flexible substrate 214. For example, the conductor interface 220 can be made of a plastic, thermoplastic, polymer, hard polymer, etc., including polyoxymethylene (e.g., DELRIN®), polyether ether ketone (PEEK), nylon, Liquid Crystal Polymer (LCP), and/or other suitable materials.

FIG. 3 illustrates a perspective view of the device 102 with the scanner assembly 110 in a rolled configuration. In some instances, the assembly 110 is transitioned from a flat configuration (FIG. 2) to a rolled or more cylindrical configuration (FIG. 3). For example, in some embodiments, techniques are utilized as disclosed in one or more of U.S. Pat. No. 6,776,763, titled “ULTRASONIC TRANSDUCER ARRAY AND METHOD OF MANUFACTURING THE SAME” and U.S. Pat. No. 7,226,417, titled “HIGH RESOLUTION INTRAVASCULAR ULTRASOUND SENSING ASSEMBLY HAVING A FLEXIBLE SUBSTRATE,” each of which is hereby incorporated by reference in its entirety.

In some embodiments, the transducer elements 212 and/or the controllers 206 can be positioned in in an annular configuration, such as a circular configuration or in a polygon configuration, around a longitudinal axis 50 of a support member 230. It will be understood that the longitudinal axis 50 of the support member 230 may also be referred to as the longitudinal axis of the scanner assembly 110, the flexible elongate member 121, and/or the device 102. For example, a cross-sectional profile of the imaging assembly 110 at the transducer elements 212 and/or the controllers 206 can be a circle or a polygon. Any suitable annular polygon shape can be implemented, such as a based on the number of controllers/transducers, flexibility of the controllers/transducers, etc., including a pentagon, hexagon, heptagon, octagon, nonagon, decagon, etc. In some examples, the plurality of transducer controllers 206 may be used for controlling the plurality of ultrasound transducer elements 212 to obtain imaging data associated with the vessel 120.

The support member 230 can be referenced as a unibody in some instances. The support member 230 can be composed of a metallic material, such as stainless steel, or non-metallic material, such as a plastic or polymer as described in U.S. Provisional Application No. 61/985,220, “Pre-Doped Solid Substrate for Intravascular Devices,” filed Apr. 28, 2014, ('220 application) the entirety of which is hereby incorporated by reference herein. The support member 230 can be a ferrule having a distal flange or portion 232 and a proximal flange or portion 234. The support member 230 can be tubular in shape and define a lumen 236 extending longitudinally therethrough. The lumen 236 can be sized and shaped to receive the guide wire 118. The support member 230 can be manufactured using any suitable process. For example, the support member 230 can be machined and/or electrochemically machined or laser milled, such as by removing material from a blank to shape the support member 230, or molded, such as by an injection molding process.

Intraluminal imaging devices, such as those illustrated in FIGS. 1-3, must be navigated through internal lumens of a patient, such as the patient's vasculature. In order to facilitate movement of the device through the internal lumens, and to reduce damage to the patient's tissue, the distal ends of imaging devices are often fitted with soft, flexible tips.

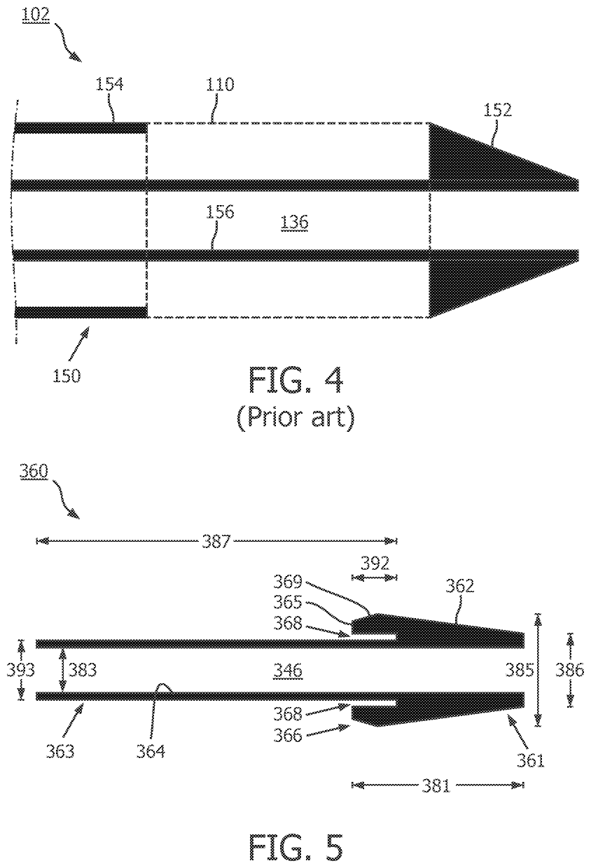

FIG. 4 depicts cross-sectional diagrammatic side view of a conventional IVUS imaging device 102 including a tip member 152. The device 102 includes an imaging assembly 110, a flexible elongate member 150 including an outer member 254 and an inner member 156, and the tip member 152 coupled to a distal end of the imaging assembly 110. The tip member 152 is coupled to the inner member 156. The tip member 152 must be secured to the inner member 156 and/or the imaging assembly 110 in a manner that ensures it will not detach in the patient's vasculature during a procedure. Thus, conventional tip members often require large amounts of adhesive to join the tip member to the other components. Thermal bonding may also be required. However, excessive amounts of adhesive in the junction between the tip member 152 and the imaging assembly 110 can disadvantageously enlarge the outer profile of the imaging device 102. Furthermore, when thermal bonding is used, the areas of the conventional tip member 152 that are bonded to the inner member 156 and/or the imaging assembly 110 are proximate the delicate electronic components of the imaging assembly 110 (e.g., ultrasound imaging elements). Thus, the geometry of conventional tip members can lead to increased risk of damage to the electronics of the imaging component from thermal bonding.

Because IVUS imaging devices often navigate confined spaces and tortuous regions of the vasculature, it is important to reduce the profile of the devices and increase flexibility without compromising the integrity of the device. Furthermore, manufacturing processes must be amenable to the delicate electronics included in the device. Accordingly, the present disclosure provides tip members that advantageously improve manufacturing and assembly processes, and improve the maneuverability of intraluminal imaging devices.

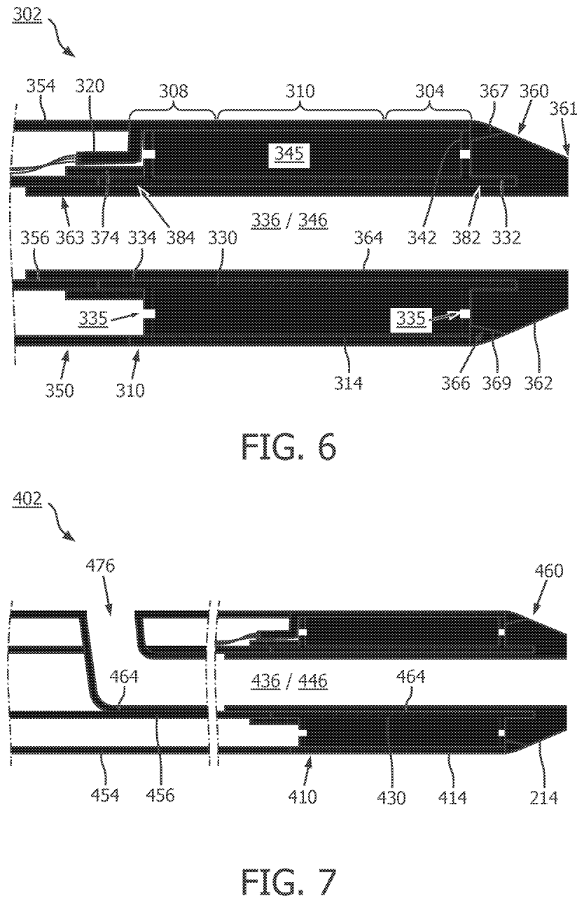

FIG. 5 is a cross-sectional diagrammatic view of a tip member 360 with an extended guidewire lumen 346, according to some aspects of the present disclosure. The tip member 360 comprises a flexible material and includes a distal guiding portion 362, a tubular extended portion 364, and an intermediate connection portion 366. The tip member 360 can comprise an integrally-formed component, such as a molded body. The guiding portion 362 is tapered down to the distal end 361 of the tip member 360, such that the guiding portion 362 comprises a conical shape. Although the outer edge of the tapered guiding portion 362 is shown in FIG. 6 as being straight, in some embodiments, the outer edge of the guiding portion 362 is curved. The tubular extended portion 364 comprises a hollow cylindrical shape, and extends proximally of the guiding portion 362 to a proximal end 363 of the tip member 360. The tubular extended portion 364 and the guiding portion 362 surround, or define, an extended guidewire lumen 336. As will be explained in greater detail below, the tubular extended portion 364 may provide other surfaces of the tip member 360 to which to bond an imaging assembly and/or a flexible elongate member of an imaging catheter, for example.

The tip member 360 further comprises an intermediate connection portion 366 at or near a proximal end of the guiding portion 362. The intermediate connection portion 366 includes a circular or annular recess or slot 368 extending distally into the guiding portion 362. The recess 368 is configured to receive a distal flange of an imaging assembly, in some embodiments. In other embodiments, the recess 368 is configured to receive a distal end of a flexible elongate member, such as a sheath, or a catheter member. In some embodiments, the recess 368 is polygonal, such as hexagonal, octagonal, or nonagonal. In other embodiments, the recess 368 comprises an elliptical shape, or any other suitable shape. The intermediate connection portion 366 also includes an intermediate shelf 365. The shelf 365 comprises a surface orthogonal to a longitudinal axis of the tip member 360 at a proximal end of the intermediate connection portion 366. The intermediate connection portion 366 also comprises an angled outer surface 369. As will be further explained below, the angled outer surface 369 may provide space for a fillet such that an outer profile of an imaging device can be minimized or maintained. Although the angled outer surface 369 is shown as being straight in FIG. 5, in other embodiments, the angled outer surface 369 may comprise a curved outer surface such that the outer surface of the guiding portion 362 of the tip member 360 maintains a smooth outer profile. In other embodiments, the intermediate connection portion 366 may not comprise an angled outer surface, such that an outer surface of the guiding portion 362 comprises a straight and/or smooth line or curve extending from the distal end 361 of the tip member 360 to the shelf 365. The flexible tip member 360 can include a variety of materials, including Pebax® and silicone.

The flexible tip member 360 can comprise a variety of dimensions of a variety of different magnitudes. For example, in some embodiments, a distal guiding portion length 381, measured from the shelf 365 to the distal end 361 of the tip member 360 can comprise a length of about 0.2 in to about 0.5 in, and between approximately 0.3 in and approximately 0.4 in, including values such as 0.30 in, 0.032 in, 0.35 in, 0.37 in, and/or other suitable values both lager and smaller. A recess length 392, measured from a proximal opening of the recess 368 to a distal end of the recess 368 can comprise a length of about 0.02 in to about 0.07 in, and between approximately 0.03 in and approximately 0.06 in, including values such as 0.040 in, 0.045 in, 0.047 in, 0.050 in, and/or other suitable values both lager and smaller. A guidewire lumen diameter 383 can comprise a diameter of about 0.005 in to about 0.03 in, and between approximately 0.01 in and approximately 0.020 in, including values such as 0.015 in, 0.016 in, 0.017 in, 0.018 in, and/or other suitable values both lager and smaller. A tubular extended portion outer diameter 393 can comprise a diameter of about 0.01 in to about 0.04 in, and between approximately 0.015 in and approximately 0.030 in, including values such as 0.018 in, 0.020 in, 0.022 in, 0.024 in, and/or other suitable values both lager and smaller. A tip member maximum outer diameter 385 can comprise a diameter of about 0.02 in to about 0.06 in, and between approximately 0.03 in and approximately 0.05 in, including values such as 0.040 in, 0.042 in, 0.044 in, 0.046 in, and/or other suitable values both lager and smaller. A distal end outer diameter 386 can comprise a diameter of about 0.01 in to about 0.03 in, and between approximately 0.010 in and approximately 0.022 in, including values such as 0.015 in, 0.017 in, 0.019 in, 0.021 in, and/or other suitable values both lager and smaller. A tubular extended portion length 387, measured from the distal end of the recess 368 to the proximal end 363 of the tip member 360, can comprise a length of about 0.2 in to about 0.6 in, and between approximately 0.3 in and approximately 0.5 in, including values such as 0.40 in, 0.42 in, 0.44 in, 0.46 in, and/or other suitable values both lager and smaller.

It will be understood that various modifications can be made to the tip member 360 contemplated by the present disclosure. For example, in some embodiments, the tip member 360 may comprise a rigid material, or a material having a variable durometer, in combination with or in lieu of the flexible material. For example, in one embodiment, the extended portion may be more rigid than the guiding portion 362, or vice versa. In some embodiments, the extended portion 364 may not be tubular. In some embodiments, the extended portion 364 may comprise any suitable shape or combination thereof, including ellipsoidal, cylindrical, circular, polygonal, and/or rectangular. The tip member 360 can be directly coupled to the imaging assembly 110, in some embodiments. In other embodiments, the tip member 360 is indirectly coupled to the imaging assembly 110. For example, in some embodiments, there are intermediate connections and connection elements used to couple the tip member 360 to the imaging assembly 110, including radiopaque markers, adhesives, ablative elements, therapeutic elements, or other suitable intermediate connecting elements.

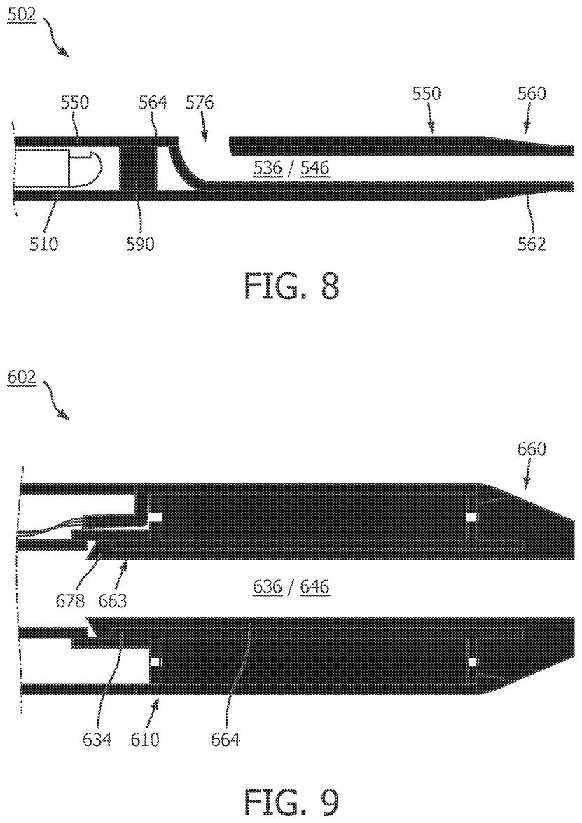

Flexible tip members with extended guidewire lumens described in the present disclosure can be included in various intraluminal imaging devices, including rotational IVUS imaging devices and solid-state IVUS imaging devices. FIGS. 6, 7, and 9, illustrate solid-state IVUS imaging devices including flexible tip members with extended guidewire lumens, and FIG. 8 illustrates a rotational IVUS imaging devices including a flexible tip member with an extended guidewire lumen.

Referring now to FIG. 6, shown there is a diagrammatic cross-sectional side view of a distal portion of an intraluminal imaging device 302, including a flexible substrate 314 and a support member 330, according to aspects of the present disclosure. The support member 330 can be referenced as a unibody in some instances. The support member 330 can be composed of a metallic material, such as stainless steel, or non-metallic material, such as a plastic or polymer as described in U.S. Provisional Application No. 61/985,220, “Pre-Doped Solid Substrate for Intravascular Devices,” filed Apr. 28, 2014, the entirety of which is hereby incorporated by reference herein. The support member 330 can be ferrule having a distal portion 382 and a proximal portion 384. The support member 330 can define a lumen 336 extending along the longitudinal axis LA. The lumen 336 is in communication with the entry/exit port 116 and is sized and shaped to receive the guide wire 118 (FIG. 1). The support member 330 can be manufactured according to any suitable process. For example, the support member 330 can be machined and/or electrochemically machined or laser milled, such as by removing material from a blank to shape the support member 330, or molded, such as by an injection molding process. In some embodiments, the support member 330 may be integrally formed as a unitary structure, while in other embodiments the support member 330 may be formed of different components, such as a ferrule and stands 342, 344, that are fixedly coupled to one another. In some cases, the support member 330 and/or one or more components thereof may be completely integrated with inner member 356. In some cases, the inner member 356 and the support member 330 may be joined as one, e.g., in the case of a polymer support member.

Stands 342, 344 that extend vertically are provided at the distal and proximal portions 382, 384, respectively, of the support member 330. The stands 342, 344 elevate and support the distal and proximal portions of the flexible substrate 314. In that regard, portions of the flexible substrate 314, such as the transducer portion 304 (or transducer region 304), can be spaced from a central body portion of the support member 330 extending between the stands 342, 344. The stands 342, 344 can have the same outer diameter or different outer diameters. For example, the distal stand 342 can have a larger or smaller outer diameter than the proximal stand 344 and can also have special features for rotational alignment as well as control chip placement and connection. To improve acoustic performance, any cavities between the flexible substrate 314 and the surface of the support member 330 are filled with a backing material 345. The liquid backing material 345 can be introduced between the flexible substrate 314 and the support member 330 via passageways 335 in the stands 342, 344. In some embodiments, suction can be applied via the passageways 235 of one of the stands 342, 344, while the liquid backing material 345 is fed between the flexible substrate 314 and the support member 330 via the passageways 335 of the other of the stands 342, 234. The backing material 345 can be cured to allow it to solidify and set. In various embodiments, the support member 330 includes more than two stands 342, 344, only one of the stands 342, 344, or neither of the stands. In that regard the support member 330 can have an increased diameter distal portion 382 and/or increased diameter proximal portion 384 that is sized and shaped to elevate and support the distal and/or proximal portions of the flexible substrate 314.

The support member 330 can be substantially cylindrical in some embodiments. Other shapes of the support member 330 are also contemplated including geometrical, non-geometrical, symmetrical, non-symmetrical, cross-sectional profiles. As the term is used herein, the shape of the support member 330 may reference a cross-sectional profile of the support member 330. Different portions the support member 330 can be variously shaped in other embodiments. For example, the proximal portion 384 can have a larger outer diameter than the outer diameters of the distal portion 382 or a central portion extending between the distal and proximal portions 382, 384. In some embodiments, an inner diameter of the support member 330 (e.g., the diameter of the lumen 336) can correspondingly increase or decrease as the outer diameter changes. In other embodiments, the inner diameter of the support member 330 remains the same despite variations in the outer diameter.

A flexible elongate member 350, including a proximal inner member 356 and a proximal outer member 354, is coupled to the proximal portion 384 of the support member 330. The proximal inner member 356 and/or the proximal outer member 354 can comprise a flexible elongate member. The proximal inner member 356 can abut a proximal flange 334. In other embodiments, the proximal inner member 356 can be received within the proximal flange 334, or the proximal flange 334 can be received within the proximal inner member 356. The proximal outer member 354 is in contact with the flexible substrate 314. In the embodiment of FIG. 6, the proximal outer member 354 is partially received within the flexible substrate 314. In other embodiments, the proximal outer member 354 can abut the substrate 314, or the substrate 314 can be received within the proximal outer member 354.

One or more adhesives can be disposed between various components at the distal portion of the intraluminal imaging device 302. For example, one or more of the flexible substrate 314, the support member 330, the tip member 360, the proximal inner member 356, and/or the proximal outer member 354 can be coupled to one another via an adhesive.

The imaging device 302 includes the flexible tip member 360 shown in FIG. 5. The flexible tip member 360 is disposed at a distal end of the imaging device 302. A tubular extended portion 364 of the tip member 360 is inserted into a lumen 336 of the imaging assembly 302. In some embodiments, the lumen 336 is a central lumen centered around a central longitudinal axis of the tip member 360. In other embodiments, the lumen 336 may be radially offset from the longitudinal axis. The tip member 360 surrounds an extended guidewire lumen 346 extending from the proximal end 363 of the tip member 360 to the distal end 361. The distal flange 332 of the imaging assembly 302 is inserted into, or received within, the annular recess 368 of the intermediate connection portion 366 of the tip member 360. The shelf 365 abuts the distal stand 342. In some embodiments, an adhesive is applied to the intermediate connection portion 366, the tubular extended portion 364, and/or the guiding portion 362, during, or prior to positioning the tip member 360 within the imaging assembly. In the embodiment of FIG. 6, an adhesive fillet 367 is deposited within a space between the angled outer surface 369 of the intermediate connection portion 366 of the tip member 360 and a distal end 361 of the imaging assembly 302. The adhesive fillet 367 can provide a seal between the tip member 360 and the imaging assembly 302 while maintaining a smooth outer profile of the imaging device 302. In that regard, the adhesive of the fillet 367 can fill one or more spaces between a surface of the distal flange 332 (e.g., exterior surface, distal surface, interior surface), an opposing surface of the tip member 360 (e.g., annular recess 368), a surface of the distal stand 342 (e.g., a distal surface), and/or a surface of the flex circuit 314 (e.g., exterior surface, distal surface). The adhesive fillet 367 can contact a plurality of such surfaces to couple the tip member 360 and the support member 330, and to seal various components of the imaging assembly 310. In other embodiments, the device 302 may not include a fillet.

The imaging assembly 302 comprises a coupling member 374 coupled to the proximal flange 334 of the imaging assembly 302. The coupling member 374 can provide a surface to couple or join one more components of the imaging assembly. The coupling member 374 may include a polymer film, such as polyimide, disposed in a cylindrical configuration around the proximal flange 334. In that regard, the coupling member 374 may be referred to as an extension tube, in some aspects. A conductor interface 320 can be coupled to an external surface of the coupling member 374. The conductor interface 320 can connect to an electrical interface of the flex circuit 314, in some embodiments. Furthermore, the proximal inner member 356 is coupled to an inner surface of the coupling member 374. The conductor interface and/or the proximal inner member 356 can be coupled to the coupling member 374 by any suitable method, including an interference fit, adhesives, and/or thermal bonding. The coupling member 374 can be coupled to the proximal flange 334 by an interference fit, adhesives, thermal bonding, and/or any other suitable coupling method.

The tubular extended portion 364 of the tip member 360 is coupled to the proximal inner member 356 at the proximal end 363 of the tip member 360 such that the proximal inner member 356 overlaps, or surrounds, the proximal end 363 of the tip member 360. The tubular extended portion 364 can be coupled to the proximal inner member 356 by any suitable method, including adhesives, thermal bonding, and/or interference fits. It will be understood that, in some embodiments, the proximal inner member 356 may not overlap or surround the tubular extended portion 364 of the tip member 360. Rather, the tubular extended portion 364 of the tip member 360 may overlap, or surround, the proximal inner member 356. In other embodiments, the proximal end 363 of the tip member 360 abuts a distal end of the proximal inner member 356. Furthermore, in some embodiments, the coupling member 374 can be coupled to the proximal flange 334 such that the outer surface of the coupling member 374 is in contact with an inner surface of the proximal flange 334.

The proximal outer member 354 is coupled to the imaging assembly 302 such that the flex circuit 314 partially overlaps the proximal outer member 354. The proximal outer member 354 can be coupled to the imaging assembly 302 by any suitable method, including adhesives and/or thermal bonding. In other embodiments, the proximal outer member 354 overlaps the flex circuit 314. In still other embodiments, the proximal outer member 354 abuts a proximal end of the flex circuit 314.

By introducing the extended tubular portion 364 of the tip member 360 into the lumen 336 of the imaging assembly 310, the tip member 360 can be coupled to the imaging assembly 310 and/or the flexible elongate member 350 at a location away from the sensitive electronic components of the flex circuit 314, such as the ultrasound transducer elements. Furthermore, little or no adhesive may be required at the junction between the distal end of the imaging assembly 310 and the tip member 360, which can help to reduce the outer profile of the imaging device 302. The coupling of the tip member 360 to the imaging assembly 310 and the proximal inner member 356 and/or proximal outer member 354 at the proximal end of the tip member 360 can provide a secure connection without increasing the outer profile of the imaging device 302, and can help reduce a risk of damage to the electronic components of the flex circuit 314. In that regard, the proximal end 363 of the tip member 360 can be thermally bonded to the imaging assembly 310 and/or the catheter outer/inner members 354, 356 to reduce the exposure of the flex circuit 314 to heat from thermal bonding.

FIG. 7 depicts a distal portion of an IVUS imaging device 402, according to another embodiment of the present disclosure. The IVUS imaging device 402 shown in FIG. 7 may comprise similar or identical components as the embodiment depicted in FIG. 6. For example, the IVUS imaging device 402 includes an imaging assembly 410 including a support member 430 and a flex circuit 414 positioned in a cylindrical configuration around the support member 430. The imaging assembly 402 is coupled to a proximal inner member 456 and a proximal outer member 454. A tip member 460 is positioned with respect to the imaging assembly 410 in a similar configuration as the embodiment of FIG. 6. However, in FIG. 7, the tip member 460 includes a tubular extended portion 464 that extends to a guidewire exit port 476. In that regard, the guidewire exit port 476 may comprise a rapid exchange port for positioning the imaging device 402 over a guidewire. Because the tubular extended portion 464 extends to the guidewire exit port 476, the tip member 460 can define an entire guidewire lumen 446. Although a proximal end 463 of the tip member 460 is shown curved outward toward the guidewire exit port 476, in some embodiments, the guidewire exit port 476 includes an opening in a wall of the tubular extended portion 464 that provides an entry/exit point into the guidewire lumen 446 within the tubular extended portion 464.

FIG. 8 depicts a rotational IVUS device 502, according to one embodiment of the present disclosure. The rotational IVUS device 502 includes an imaging assembly 510 disposed within an outer sheath 550. The device 502 includes a flexible tip member 560 coupled to a distal portion 551 of the outer sheath 550. Similar to the embodiments shown in FIGS. 6 and 7, the tip member 560 includes a guiding portion 562, a tubular extended portion 564, and an intermediate connection portion 566. The intermediate connection portion 566 comprises an annular recess 568 extending into the guiding portion 562, with a distal end 553 of the outer sheath 550 disposed within the annular recess 568. The tubular extended portion 564 extends to, or beyond, a guidewire exit port 576. The guidewire exit port 576 comprises an opening in the outer sheath 550 to the extended guidewire lumen 546 in the tubular extended portion 564 to facilitate insertion and removal of a guidewire. In that regard, the guidewire exit port 576 can comprise a rapid exchange port. The tip member 560 is disposed distally of the imaging assembly 510 within the outer sheath 550. In the embodiment of FIG. 8, the device 502 comprises a sealing member 590 disposed within the outer sheath 550 distal of the imaging assembly 510 to provide a fluid seal between the imaging assembly 510 and the extended guidewire lumen 546.

FIG. 9 depicts an IVUS imaging device 602, according to another embodiment of the present disclosure. In the embodiment of FIG. 9, the molded tip member 660 includes a proximal attachment portion 678 extending radially outward from the tubular extended portion 664 at the proximal end 663 of the tip member 660. In that regard, the proximal attachment portion 678 can be described as a radial projection, in some aspects. The proximal attachment portion 678 engages the proximal flange 634 of the imaging assembly 610. In some embodiments, the proximal attachment portion 678 can secure the tip member 660 to the imaging assembly 610 without the need for other coupling methods (e.g., adhesives, thermal bonding). In other embodiments, the proximal attachment portion 678 is used in conjunction with adhesives, thermal bonding, and/or any other suitable coupling method to secure the tip member 660 to the imaging assembly 610 and/or the flexible elongate member 650.

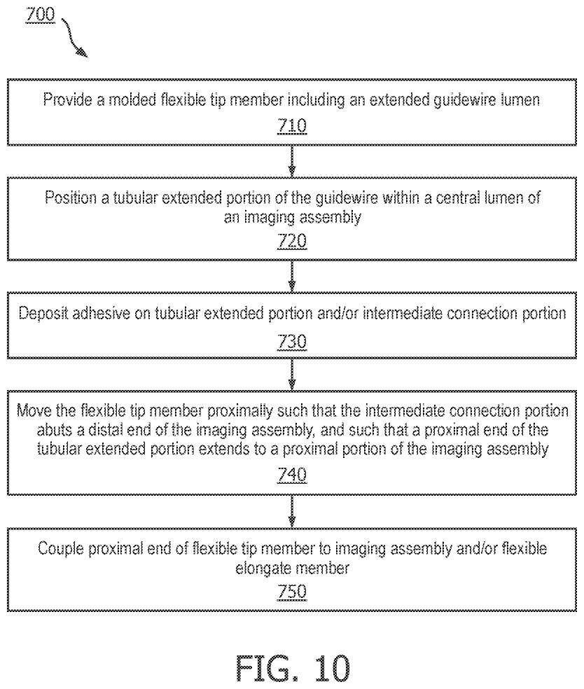

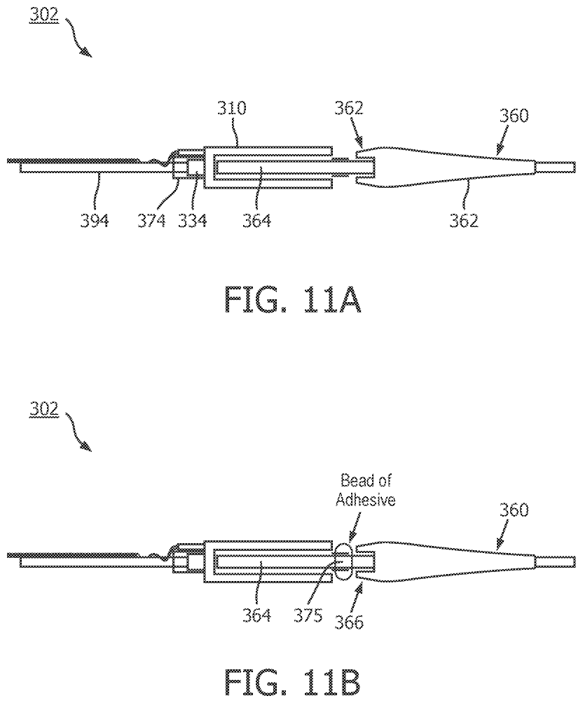

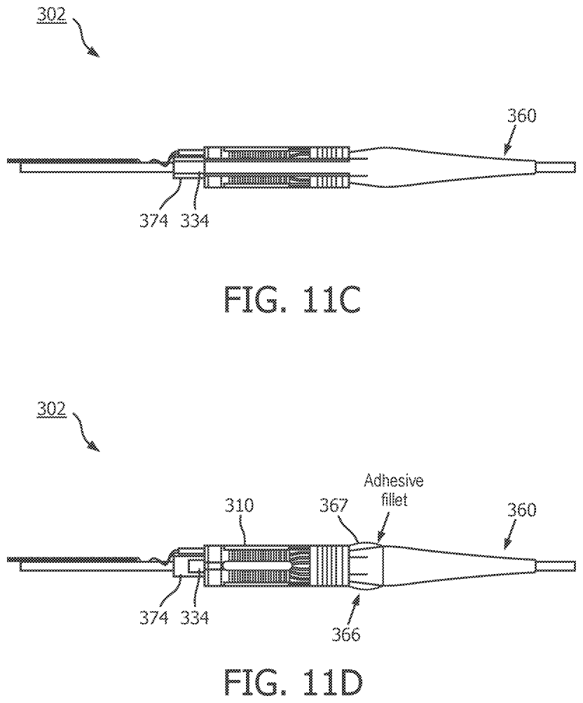

FIG. 10 is a flow chart illustrating a method 700 for assembling an intraluminal imaging device 302 with a flexible tip member 360 comprising an extended guidewire lumen, according to some embodiments of the present disclosure. For example, the method 700 may be performed using the flexible tip member 360 and imaging device 302 shown in FIG. 6. The steps of the method 700 are also illustrated in corresponding FIGS. 11A-11D In step 710 of the method 700, a flexible tip member 360 is provided comprising a molded (e.g., integrally formed) body that includes a guiding portion 362, an intermediate connection portion 366, and a tubular extended portion 364. The guiding portion 362 may be tapered such that a diameter of the guiding portion 362 decreases from the proximal portion of the guiding portion 362 to the distal portion of the guiding portion 362. The tubular extended portion 364 may extend proximally of the guiding portion 362. The intermediate connection portion 366 can include a recess configured to receive a distal portion of an IVUS imaging assembly 310, such as the recess 368 shown in FIG. 5, and a shelf configured to abut a distal end of the imaging assembly.

In step 720, also shown in FIG. 11A, the tubular extended portion 364 of the tip member 360 is at least partially inserted into a lumen of an imaging assembly 310. In other embodiments, such as the rotational IVUS embodiment of FIG. 8, the tubular extended portion can be inserted into a flexible elongate member. As shown in FIG. 11A, the flexible tip member 360 and imaging assembly 310 are positioned over, or around, an assembly mandrel 394. The Assembly mandrel 394 is used during assembly, and then removed. At another point during assembly, an inner catheter member and/or outer catheter member are coupled to imaging assembly 310. A gap is left between the distal end of the imaging assembly 310 and the shelf of the intermediate connection portion 366 for adhesive. In step 730, shown in FIG. 11B, a bead of adhesive 375 is applied to the tubular extended portion 364, a distal flange of the imaging assembly 310, and a surface of the intermediate connection portion 366. In step 740, shown in FIG. 11C, the flexible tip member 360 is moved proximally such that the intermediate connection portion 366 (e.g., the shelf) abuts the distal end of the imaging assembly 310, and such that a proximal end of the tubular extended portion 364 extends to a proximal portion of the imaging assembly 310. As mentioned above, the intermediate connection portion 366 may comprise a recess configured to receive the distal flange of the imaging assembly 310 or a distal end of a flexible elongate member (e.g., a sheath). In that regard, step 740 can include inserting the distal flange of the imaging assembly 310 or the distal end of the flexible elongate member into the recess.

In step 750, also shown in FIG. 11C, a proximal end of the flexible tip member 360 is coupled to the imaging assembly 310 and/or a flexible elongate member. For example, in a solid-state IVUS device, step 750 can include coupling the proximal end of the flexible tip member 360 to a distal end of a proximal inner member, a proximal flange of an IVUS imaging assembly, and/or the coupling member 374 coupled to the proximal flange 334 of the IVUS imaging assembly. The flexible tip member 360 can be coupled to the imaging assembly 310 and/or the flexible elongate member by adhesives, thermal bonding, or any other suitable coupling method. For example, as shown in FIG. 9, the flexible tip member 660 can include a proximal attachment member 678 configured to engage a proximal surface of the imaging assembly 610, such as a proximal flange 634.

As shown in FIG. 11D, in some embodiments, the method 700 can also include applying an adhesive fillet 367 around the intermediate connection portion 366 of the flexible tip member 360 to provide a smooth outer profile of the device 302 and to seal various components of the imaging assembly 310.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An intraluminal imaging device, comprising:
a flexible elongate member configured to be positioned within a body lumen of a patient, the flexible elongate member comprising a proximal portion and a distal portion;
an imaging assembly coupled to the distal portion of the flexible elongate member, the imaging assembly surrounding a lumen; and
a tip member coupled to the imaging assembly, the tip member comprising a molded body including a guiding portion and an extended portion,
wherein the guiding portion extends distally of the imaging assembly,
wherein the extended portion extends proximally of the guiding portion through the lumen within the imaging assembly, and
wherein the tip member comprises a guidewire lumen extending through the guiding portion and the extended portion.

2. The intraluminal imaging device of claim 1, further comprising:
an adhesive fillet positioned around an external surface of a proximal portion of the guiding portion, the adhesive fillet contacting a distal end of the imaging assembly such that the fillet seals a junction between the guiding portion of the tip member and the distal end of the imaging assembly.

3. The intraluminal imaging device of claim 1, wherein the imaging assembly comprises an intravascular ultrasound (IVUS) imaging assembly, the IVUS imaging assembly comprising a flexible substrate positioned around a support member.

4. The intraluminal imaging device of claim 3, wherein the imaging assembly comprises an extension tube attached to a proximal flange of the support member, and wherein a proximal end of the extended portion of the tip member is attached to the extension tube.

5. The intraluminal imaging device of claim 4, wherein the flexible substrate comprises an electrical interface disposed at a proximal end of the flexible substrate, the electrical interface secured to an outer surface of the extension tube.

6. The intraluminal imaging device of claim 3, wherein the tip member comprises an intermediate connection portion between the guiding portion and the extended portion, the intermediate connection portion comprising a recess extending distally into the guiding portion, wherein a distal flange of the support member is received within the recess.

7. The intraluminal imaging device of claim 1, wherein the flexible elongate member comprises a guidewire exit port, and wherein the extended portion extends proximally within the flexible elongate member to the guidewire exit port, such that the guidewire lumen extends from the guidewire exit port to a distal end of the tip member.

8. The intraluminal imaging device of claim 1, wherein the flexible elongate member comprises a proximal inner member and a proximal outer member, and wherein a proximal end of the extended portion of the tip member is coupled to a distal end of the proximal inner member.

9. The intraluminal imaging device of claim 1, wherein the extended portion comprises a radial projection at a proximal end of the extended portion of the tip member, the radial projection configured to engage a proximal surface of the imaging assembly to mechanically secure the tip member to the imaging assembly.

10. The intraluminal imaging device of claim 1, wherein the guiding portion of the tip member comprises a tapered tubular shape comprising a first outer diameter at a proximal end of the guiding portion and a second outer diameter at a distal end of the guiding portion, and wherein the extended portion comprises a non-tapered shape comprising a third outer diameter, wherein the first outer diameter is larger than the second outer diameter and the third outer diameter.

11. A method for manufacturing an intraluminal imaging device, comprising:
providing a tip member comprising a molded body including a guiding portion, an extended portion extending proximally of the guiding portion, and an intermediate connection portion disposed at a junction of the guiding portion and the extended portion;
positioning the extended portion within a lumen of an imaging assembly;
applying adhesive on or near the intermediate connection portion; and
moving the tip member proximally such that the intermediate connection portion abuts a distal end of the imaging assembly, and such that a proximal end of the extended portion extends to a proximal portion of the imaging assembly.

12. The method of claim 11, wherein applying the adhesive comprises forming an adhesive fillet on an exterior surface of the intermediate connection portion such that the fillet provides a seal between the guiding portion of the tip member and the imaging assembly.

13. The method of claim 11, wherein the intermediate connection portion of the tip member comprises a recess extending distally into the guiding portion, and wherein moving the tip member proximally comprises inserting a distal flange of the imaging assembly into the recess.

14. The method of claim 11, wherein moving the tip member proximally comprises positioning a proximal end of the extended portion adjacent a proximal flange of the imaging assembly.

15. The method of claim 14, further comprising at least one of thermally or adhesively bonding an extension around the proximal flange of the imaging assembly and the proximal end of the extended portion.

16. The method of claim 11, further comprising coupling a distal end of a flexible elongate member to the imaging assembly, wherein moving the tip member proximally comprises positioning a proximal end of the extended portion at a guidewire exit port of the flexible elongate member.
